(19) United States
(12) Patent Application Publication
(10) Pub. No.: US 2025/0186317 A1
(43) Pub. Date: Jun. 12, 2025

(54) SKIN CARE COMPOSITION COMPRISING LACTIC ACID, POLYHYDROXY ACID, AND ITS BUFFERING SALT

(71) Applicant: The Procter & Gamble Company, Cincinnati, OH (US)

(72) Inventors: Matthew Clair Ehrman, Singapore (SG); Wan Ting Chung, Singapore (SG); Sudeep Chakravarty, Singapore (SG); Xi Ping Ng, Singapore (SG)

(21) Appl. No.: 18/971,160

(22) Filed: Dec. 6, 2024

Related U.S. Application Data
(60) Provisional application No. 63/666,276, filed on Jul. 1, 2024.
(60) Provisional application No. 63/606,790, filed on Dec. 6, 2023.

(51) Int. Cl. A61K 8/365; A61Q 19/00

(52) U.S. Cl. CPC A61K 8/365; A61Q 19/00

(57) ABSTRACT

A low-pH, aqueous skin care composition that contains lactic acid, a polyhydroxy acid (PHA), a buffering salt of the polyhydroxy acid (PHA), and water. The pH of the composition is from about 2.5 to 4.5. The composition provides reduced skin irritation and/or improving skin barrier function, while providing skin care efficacy/improving skin condition and/or providing stability of the composition.

### FIELD

Disclosed is a low-pH, aqueous skin care composition, comprising: (a) Lactic acid; (b) a polyhydroxy acid (PHA); (c) a buffering salt of the polyhydroxy acid (PHA); and (d) water, wherein the pH of the composition is from about 2.5 to 4.5, wherein the composition has a specific weight ratio among at least two of the three ingredients (a), (b) and (c). The composition provides reduced skin irritation and/or improving skin barrier function, while providing skin care efficacy/improving skin condition and/or providing stability of the composition.

### BACKGROUND

Skin is the first line of defense against environmental insults that would otherwise damage sensitive underlying tissue and organs. Additionally, skin plays a key role in a person's physical appearance. Not surprisingly, most people would like to have heathy, younger looking skin. Unfortunately for some people, the tell-tale signs of aging such as thinning skin, wrinkles, and age spots are an undesirable reminder of the disappearance of youth. The desire for healthy, younger looking skin has led to the development of numerous skin care products marketed to treat the various skin conditions associated with aging and unhealthy skin. These skin care products typically include one or more active ingredients for treating a skin condition of interest.

Such skin care compositions can be formulated as low-pH compositions. For example, WO2023097321 discloses a low-pH composition comprising hydroxycinnamic acid (HCA), polar emollient, and co-solvent for HCA. WO2023097321 also discloses that “The buffering agent may be selected according to the acid(s) that is used to lower the pH of the low-pH compositions herein. For example, lactic acid and gluconic acid may be used, individually or in combination, to lower the pH of the composition because they are generally considered gentler on skin (i.e., lower risk of irritation) compared to other alpha hydroxy acids. In this example, sodium lactate and/or sodium gluconate would then be selected as the corresponding salt buffering agent to provide the acid/salt pH buffer system.” The present inventors have found a need to further improve the buffering system, for example, for further reducing skin irritation and/or improving skin barrier function (such as transepidermal water loss (TEWL)), while providing skin care efficacy/improving skin condition and/or providing stability of the composition.

### SUMMARY

Disclosed herein is a low-pH, aqueous skin care composition, comprising:
(a) Lactic acid;
(b) A polyhydroxy acid (PHA);
(c) A buffering salt of the polyhydroxy acid (PHA); and
(d) Water,
wherein the pH of the composition is from about 2.5 to 4.5,
wherein the composition meets at least one of the following conditions (i)-(iii):
(i) wherein the weight ratio between “the sum of (b) PHA and (c) PHA buffering salt” and (a) lactic acid is from about 14:1 to about 1:1;
(ii) wherein the weight ratio between (b) PHA and (a) Lactic acid is from about 10:1 to about 2:1;
(iii) wherein the weight ratio between (c) PHA buffering salt and (a) Lactic acid is from about 10:1 to about 2:1.

The present inventors have surprisingly found increased irritations in lactic acid buffering system, especially when the buffering system is used in the following conditions: at low pH; under UV radiation; with polar emollients; with HCA; and/or with co-solvents of HCA. The present inventors have also surprisingly found that when gluconic acid is used as the buffering system without Lactic acid, the composition tend to have reduced stability and/or reduced skin care efficacy/reduced improvement of skin condition. The present inventors have further surprisingly found that reduced stability (especially photo stability) of the composition when lactic acid buffering system is used together with mica.

The present inventors have surprisingly found that, by the use of the specific buffering system comprising (a) Lactic acid, (b) A polyhydroxy acid (PHA), and (c) A buffering salt of the polyhydroxy acid (PHA) at a specific weight ratio among at least 2 out of these 3 ingredients, the composition provides reduced skin irritation and/or improved skin barrier function (such as transepidermal water loss (TEWL)), while providing skin care efficacy/improving skin condition and/or providing stability of the composition, especially in the following conditions: at low pH; under UV radiation; with polar emollients; with HCA; with co-solvents of HCA; and/or with mica.

### DETAILED DESCRIPTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application,” as used in reference to a composition, means to apply or spread the compositions onto a human skin surface such as the epidermis.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, an effective amount of a vitamin B₃ compound is an amount sufficient to improve the health and/or appearance of psoriatic skin during a treatment period. In some instances, an effective amount may be demonstrated using ex vivo and/or in vitro methods.

“Hydroxycinnamic acid” (HCA) refers to a class of aromatic acids or phenylpropanoids having a C6-C3 skeleton that are hydroxy derivatives of cinnamic acid. Some non-limiting examples of HCA are caffeic acid, cichoric acid, cinnamic acid, chlorogenic acid, diferulic acids, coumaric acids (p-, o-, and m-isomers), ferulic acid, sinapinic acid, sinapic acid, and α-cyano-4-hydroxycinnamic acid.

“Improve the appearance of” means providing a measurable, desirable change or benefit in skin appearance, which may be quantified, for example, by a decrease in redness, inflammation, and/or plaque scales.

“Low-pH” means a pH of less than 5.0 (e.g., 1.5 to 4.9, 2.0 to 4.5, 2.5 to 4.0, or 3.5 to 4.0). A suitable method of determining the pH of a composition is described in more detail below.

“Neutral pH” means a pH of 5.0 to 8.0.

“Safe and effective amount” means an effective amount of an ingredient that is low enough to avoid serious side effects (within the scope of sound medical judgment).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Composition

The composition disclosed herein is a low-pH, aqueous skin care composition, comprising:
(a) Lactic acid;
(b) A polyhydroxy acid (PHA);
(c) A buffering salt of the polyhydroxy acid (PHA); and
(d) Water,
wherein the pH of the composition is from about 2.5 to 4.5,
wherein the composition meets at least one of the following conditions (i)-(iii):
(i) wherein the weight ratio between “the sum of (b) PHA and (c) PHA buffering salt” and (a) lactic acid is from about 14:1 to about 1:1, preferably from about 12:1 to about 2:1, more preferably from about 10:1 to about 5:1;
(ii) wherein the weight ratio between (b) PHA and (a) Lactic acid is from about 10:1 to about 2:1, preferably from about 7:1 to about 2:1, more preferably from about 5:1 to about 3:1; and
(iii) wherein the weight ratio between (c) PHA buffering salt and (a) Lactic acid is from about 10:1 to about 2:1, preferably from about 7:1 to about 2:1, more preferably from about 5:1 to about 2.5:1.

The skin care compositions described herein are low-pH compositions intended for topical application to human skin to improve the appearance, health, and/or function of skin. The present compositions may be used for non-therapeutic (i.e., cosmetic) treatment of a variety of skin conditions. For example, the low-pH composition may be particularly suitable for improving the appearance of skin, especially fine lines, wrinkles, hyperpigmented spots, uneven skin tone, sallow looking skin, and/or skin stability. Improved skin stability means to provide reduced day-to-day fluctuation of skin condition.

The low-pH skin care compositions herein can be made by mixing the ingredients with a dermatologically acceptable carrier using conventional methods known to those skilled in the art. The compositions may be provided in various product forms such as solutions, suspensions, lotions, creams, gels, toners, sticks, sprays, aerosols, ointments, cleansing liquid washes and solid bars, pastes, foams, mousses, shaving creams, wipes, strips, patches, electric-powered patches, hydrogels, film-forming products, facial and skin masks (with and without insoluble sheet), and the like. The composition form may follow from the particular dermatologically acceptable carrier chosen.

In some instances, the low-pH skin care composition herein may be in the form of an essence. An essence is a form of topical skin care composition in a relatively concentrated formula that typically has a lower viscosity than a conventional cream- or lotion-type skin care composition. An essence may be provided in the form of a low viscosity fluid that is marketed to specifically target a particular skin condition and/or be used in the first step of a skin care regimen. The skin care essence products herein can have a dynamic viscosity of 1 centipoise (cP) to 15,000 cP at 25° C. (e.g., 50 cP to 10,000 cP or 100 cP to 7,500 cP, 200 cp to 5,000 cp, or 300 cp to 2,500 cp). A method of determining the viscosity of the low-pH compositions is described in more detail in the Methods section below.

### Low-pH Acid Buffering System

When providing a low-pH composition for topical application to skin, it can be important to include a buffering system to help maintain the pH of the composition for a period of time after it is applied to the skin (e.g., up to 5 minutes or more). On average, human skin pH typically ranges from about 5.0 to 6.0. To maintain this pH, human skin has evolved a natural buffering system that resists changes to pH. Thus, when a low-pH composition is applied to the skin, the skin's natural buffering system will try to adjust the pH of the composition to match the natural pH of the skin. Without the addition of the buffering agent, the low-pH composition may not be able to provide the desired skin care benefit. Accordingly, the compositions herein may include a low-pH acid buffering system.

### Lactic Acid/Alpha Hydroxy Acid

The composition comprises Lactic acid or Alpha hydroxy acid.

Alpha hydroxy acid useful herein include, lactic acid, citric acid, glycolic acid, and mandelic acid. Among them, lactic acid is preferred because it's generally considered gentler on skin (i.e., lower risk of irritation) compared to other alpha hydroxy acids.

Lactic acid/Alpha hydroxy acid can be included in the composition at a level by weight of the composition of from about 0.05% to about 1%, preferably from about 0.05% to about 0.75%, more preferably from about 0.3% to about 0.75%.

### A Polyhydroxy Acid (PHA)

The composition comprises a polyhydroxy acid (PHA). PHA useful herein is selected from the group consisting of, for example, Gluconic acid and its precursors like Glucono-deltalactone (GDL), and its derivatives like disaccharide formed from gluconic acid and galactose, Lactobionic acid, Maltobionic acid, and mixtures thereof. Gluconic acid is formed through the hydrolysis of Glucono-deltalactone (GDL). Without being limited theory, among them, preferred are Gluconic acid and Glucono-deltalactone (GDL), and more preferred is Glucono-deltalactone (GDL), because they may be generally considered to be more stable than the lactobionic acid and maltobionic acid with regards to color stability at higher temperatures.

PHA can be included in the composition at a level by weight of the composition of from about 0.1% to about 5%, preferably from about 0.5% to about 4, more preferably from about 1% to about 3.5%

### A Buffering Salt of the Polyhydroxy Acid (PHA)

The composition comprises a buffering salt of the polyhydroxy acid (PHA). Such buffering salts are, for example, sodium salts, calcium salts, potassium salts of the above PHA. Among them, preferred are sodium salts, and more preferred is Sodium Gluconate.

The buffering salt may be present in the low-pH composition at a level by weight of the composition, of from about 0.1% to about 5%, preferably from about 0.5% to about 4%, more preferably from about 01% to about 3%.

### Hydroxycinnamic Acid

The low-pH skin care compositions herein may include a safe and effective amount of an HCA. The HCA may be present in the composition at 0.1% to 10% by weight of the composition (e.g., 0.5% to 5% or 1% to 4%). Hydroxycinnamic acids are generally recognized as antioxidant phenolic compounds, which can be found in plants, mainly as a component of cell walls. See, H. K. Kuzaki et al., J. Agric. Food Chem., 50, 2161-68 (2002). In some aspects, it may be desirable to select coumaric acid for use in the low-pH composition, especially p-coumaric acid (a.k.a. 4-HCA). 4-HCA has the formula:

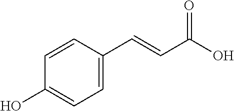 [SMILES: O=C(O)/C=C/c1ccc(O)cc1]

A particularly suitable example of an HCA material suitable for use herein is LIPOBRITE available from Vantage Personal Care.

### Polar Emollient and a Co-Solvent

The low-pH compositions herein may contain a polar emollient and/or a co-solvent with a Hansen solubility parameter distance of less than 15 from the HCA, especially when the composition contains HCA. In an aqueous, low-pH composition, HCA has a tendency to precipitate out of the composition and form crystals (HCA crystals), which can undesirably affect the look, feel, and/or efficacy of the composition. Conventional low-pH compositions often contain HCA crystals. The above ingredients, especially the combination of the polar emollient and the co-solvent greatly improve the solubility of HCA in a low-pH aqueous composition and reduce or inhibit HCA crystallization/precipitation.

In some aspects, the low-pH compositions herein may include from about 0.1% to about 10% of a polar emollient by weight of the composition, and preferably such that the weight ratio between HCA and the polar emollient is from about 1:1 to about 1:10.

In some aspects, the low-pH compositions herein may include 0.5% to 7% of a polar emollient by weight of the composition (e.g., 1% to 5% ELDEW SL-205 brand isopropyl lauroyl sarcosinate available from Ajinomoto OmniChem), which has been found to solubilize p-coumaric acid, especially when combined with a suitable glycol. Some non-limiting examples of polar emollients that may be suitable for use herein are amino acid ester derivatives (e.g., lauroyl arginine, arginine lauroyl glutamate, phytosteryl/octyldodecyl lauroyl glutamate, and dihexyldecyl lauroyl glutamate) and hydrocarbon esters (e.g., cyclohexylglycerin and isopropyl myristate).

The compositions herein also include a co-solvent with a Hansen solubility parameter distance of less than 15 from the HCA that acts in combination with the polar emollient to help solubilize the HCA. The Hansen solubility parameter distance is described in Hansen solubility parameters: a user's handbook, CRC press, 2007 and can be calculated using the following equation:

$$\sqrt{4 * {( {δ}_{D 2} - {δ}_{D 1} )}^{2} + {( {δ}_{p 2} - {δ}_{p 1} )}^{2} + {( {δ}_{H 2} - {δ}_{H 1} )}^{2}}$$

Where:
   δ_{D}=Hansen dispersion,
δ_{P}=Hansen polarity, and
δ_{H}=Hansen hydrogen bonding.

Co-solvents suitable for use herein include short chain dihydric alcohols (e.g., glycols). However, when a glycol is selected as the co-solvent, it can be important to limit the amount of glycol in the low-pH composition to less than 25% by weight of the composition (e.g., less than 20%, less than 17%, less than 15%, or even less than 10%), but generally greater than 1%, to avoid undesirable feel characteristics (e.g., sticky feeling or greasy feeling). Some non-limiting examples of glycols that may be suitable for use herein are propylene glycol, dipropylene glycol, butylene glycol, pentylene glycol, hexylene glycol, ethoxydiglycol, and C2-C6 polyethene glycols (e.g., PEG-3, PEG-4, PEG-4 methyl ether), and combinations thereof.

In some aspects, the polar emollient and co-solvent may be present in the low-pH composition at a weight ratio of polar emollient to co-solvent of 1:3 to 3:1 (e.g., 2:3, 1:1, or 2:1). In some aspects, the HCA may be premixed with the co-solvent and/or polar emollient and, optionally, one or more other ingredients and then added to the composition.

### Sensory Improving Ingredients

The composition may further contain sensory improving agent, especially when the composition contains higher amounts of polar emollients, co-solvents, and mixtures thereof.

When the compositions contain higher amount of polar emollient, co-solvent, and mixtures thereof, such compositions may have an unpleasant consumer sensory experience for various attributes, including stickiness, oiliness, greasiness, coated residue feeling, shininess, and lack of absorption into skin. Thus, in such compositions, it is preferred to add sensory improvement ingredients. Such sensory improving ingredients useful herein included: sugar alcohols such as trehalose and xylitol; sugar-based emulsifiers such as sucrose dilaurate, and sorbitan stearate. Surprisingly, it was found these materials which were known to provide negative sensory perception such as stickiness, actually counter-acted the polar emollient and co-solvent effect in this case. Among them, sugar alcohols are more preferred in view of providing additional skin conditioning benefit together with sensory improving benefit.

It is preferred that the sensory improving ingredient is included in the composition at a level of from about 0.1% to about 5% by weight of the composition. It is also preferred that the weight ratio between sensory improving ingredient and co-solvent is from about 2:1 to about 1:20, more preferably from about 1.5:1 to about 1:10, still more preferably from about 1.5:1 to about 1:5.

### Vitamin B₃ Compound

The present composition may include a safe and effective amount of a vitamin B₃ compound for regulating a variety of skin condition, for example, as described in U.S. Pat. No. 5,939,082. The compositions herein may contain 0.1% to 10%, by weight, of the vitamin B₃ compound, based on the weight or volume of the composition (e.g., 0.5% to 5% or 1% to 4%).

As used herein, “vitamin B₃ compound” means a compound having the formula:

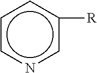

Where:

R is CONH₂ (i.e., niacinamide), COOH (i.e., nicotinic acid) or CH₂OH (i.e., nicotinyl alcohol); derivatives thereof; and salts of any of the foregoing. Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide. In some instances, vitamin B₃ compounds such as niacinamide may have improved efficacy at lower pH, for example, as described in U.S. Publication No. 2020/0009123.

In some instances, it may be desirable for the ring nitrogen of the vitamin B₃ compound to be “uncomplexed” (e.g., chemically unbound and/or unhindered) in the composition and/or prior to application to a target skin surface. For example, the compositions herein may be free of or substantially free of (i.e., less than 3%, 2%, 1% or even less than 0.5%) a salt or complex of a vitamin B₃ compound. Exemplary approaches to minimizing or preventing the formation of undesirable salts and/or complexes include omission of materials that form substantially irreversible or other undesirable complexes with the vitamin B₃ compound in the composition, pH adjustment, ionic strength adjustment, the use of surfactants, and practicing formulation processes wherein the vitamin B2 compound and materials which complex therewith are in different phases.

### Dermatologically Acceptable Carrier

The low-pH compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition. Preferably, the dermatologically acceptable carrier is water.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion. The emulsion may have a continuous aqueous phase (e.g., an oil-in-water or water-in-oil-in-water emulsion) or a continuous oil phase (e.g., water-in-oil or oil-in-water-in-oil emulsion). The oil phase may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable, hydrophilic diluents. As used herein, “diluent” includes materials in which the vitamin B₃ compound can be dispersed, dissolved, or otherwise incorporated. Hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Mica

The composition may further comprise mica at a level of from about 0.01% to about 5%, preferably from about 0.05% to about 3%, more preferably from about 0.1% to about 2% by weight of the composition.

It is preferred that the mica has a mean particle size of from about 5 microns to about 20 microns, more preferably from about 7 microns to about 17 microns, still more preferably from about 10 microns to about 17 microns.

The mica useful herein can be natural and/or synthetic, for example, synthetic mica may have an INCI name of Fluorophlogopite. The mica useful herein are thin and plate-like particles and can be a base substrate particle layered with film of TiO2, with or without additives of tin oxide, wherein the thickness of the layers is from about 50 nm to about 300 nm. Such mica layered with TiO2 can be further coated by iron oxide. Mica with or without layered films of TiO2 etc. has an aspect ratio of, preferably at least 5, more preferably more than 10, still more preferably more than 20. The aspect ratio can be determined by the average thickness and the average length (also called as average particle size) according to the formula: aspect ratio=length/thickness.

An example of mica useful herein is a synthetic mica in view of reducing friction/draggy feel and in view of controlling heavy metal contents. The mica can also be layered with films of TiO2 is used herein.

The mica useful herein may comprise an additional surface treatment to reduce interactions with other materials, to modify the skin feel, and/or to improve dispersibility. For example, the surface may be hydrophobically modified to increase suspension in a hydrophobic phase or composition. The surface-treated interference pigments may have a coating comprising from about 0.1% to about 20%, alternatively from about 0.1% to about 10%, and alternatively from about 0.1% to about 3% of the total particulate weight. Nonlimiting examples of surface treatments useful herein include silicones, acrylate silicone copolymers, acrylate polymers, alkyl silane, isopropyl titanium tri-isostearate, sodium stearate, magnesium myristate, perfluoroalcohol phosphate, perfluoropolymethyl isopropyl ether, lecithin, carnauba wax, polyethylene, chitosan, plant lipid extracts and mixtures thereof, preferably, silicones, silanes, stearates and mixtures thereof.

### TRPV Inhibitor

The composition may further contain from about 0.1% to about 2%, preferably from about 0.25% to about 1.5%, from about 0.5% to about 1.5% of an additional TRPV1 inhibitor by weight of the composition. Such additional TRPV1 inhibitor is preferably 4-t-Butylcyclohexanol, for example, that supplied with a tradename Symsitive.

### Thickener

The composition may include a polymer thickener that can tolerate a low-pH, electrolytic environment. That is, the thickener will not lose its ability to thicken or stabilize the composition at low-pH in the presence of an acid-salt buffering system. Some conventional neutralized thickeners are known to degrade and/or lose the ability to suitably thicken a composition at lower pH and/or in the presence of an acid-salt buffer (e.g., sodium lactate). For example, some neutralized thickeners degrade in a low-pH environment. On the other hand, fatty alcohol thickeners such as cetyl alcohols and stearyl alcohols are generally stable at low pH, but tend to impart an undesirable cloudiness or opacity to the composition when it is in the form of an essence, serum, or the like. It has also been found that certain anionic polymeric thickeners can provide suitable tolerance to low-pH environments but cannot tolerate buffer systems due to combination of acid and salt. Thus, in some instances, the low-pH composition described herein may be free or substantially free of neutralized thickeners, fatty alcohol thickeners, and anionic thickeners. The thickener may be present at 0.0001% to 25% (e.g., 0.001% to 20%, 0.01% to 10%, 0.5% to 7%, or 1% or 5%) by weight of the composition.

Other nonlimiting examples of thickeners or water structuring agents that may be used alone or in combination herein include natural or synthetic gums, polysaccharides, carboxylic acid polymers, polyacrylamide polymers, sulfonated polymers, and copolymers of these. Further examples include modified gums, celluloses, and superabsorbent polymers. The term “superabsorbent polymer” is understood to mean a polymer which is capable, in its dry state, of spontaneously absorbing at least 20 times its own weight of aqueous fluid, in particular of water and especially of distilled water. Suitable polysaccharides include alkyl hydroxyalkyl cellulose ethers, such as hydroxypropylmethylcellulose stearoxy ether. This material is sold under the tradename of SANGELOSE 60L and 90L from Daido Chemical Corp. Another suitable polysaccharide includes hydrophobically modified starch, such as Potato modified starch. This material is sold under the tradename of STRUCTURE SOLANACE by Nouryon. Another polymer includes crosslinked polymers, the monomers of which are at least partially composed of acryloyldimethyltaurate monomers, such as, for example sodium polyacryloyldimethyl taurate, sold under the tradename of ARISTOFLEX SILK, from Clariant.

It has now been found that certain anionic polymeric thickeners can provide suitable tolerance to low-pH environments and the desired feel and opacity properties to the composition. Thus, a particularly suitable example of an anionic thickener is polyacrylate crosspolymer-6, which is commercially available as SEPIMAX ZEN from Seppic, France.

### Low Molecular Weight Silicone Fluid

In some instances, an anionic polymeric thickener may impart an undesirable tacky feel when the low-pH composition is applied to a target portion of skin. It has been found that the addition of a low molecular weight silicone fluid can reduce or prevent this tacky feel. The molecular weight of a silicone fluid depends on the length of its silicone polymer chain(s), which is also directly proportional to the viscosity of the silicone fluid. Thus, the low molecular weight silicone fluids suitable for use in the present low-pH composition have a kinematic viscosity of 100 cSt or less at 25° C. (e.g., 1 cSt to 90 cSt, 5 cSt to 50 cSt, or even 10 cSt to 30 cSt). Kinematic viscosity is a common method of classifying silicone fluids and can be obtained from the supplier of the material. A particularly suitable example of a low molecular weight silicone fluid is 5 cSt dimethicone fluid. As used herein, “dimethicone” means a polydimethylsiloxane compound having the formula:

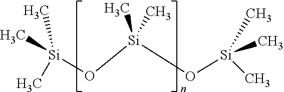

The low molecular weight silicone fluids may be present in the composition at 0.1% to 10% by weight of the composition.

### Antioxidant

The low-pH composition herein may include an antioxidant, especially when the composition includes HCA, to provide improved HCA stability. The low-pH compositions herein are also formulated to HCAs are relatively good antioxidant materials, and thus tend to be oxidized and/or degrade over time, which can result in a skin care composition that exhibits an undesirable color change (e.g., yellowing), an undesirable odor, and/or reduced efficacy. Formulating at a lower pH can improve HCA stability by reducing the rate at which it is oxidized, but it may be desirable, in some aspects, to include an antioxidant in the low-pH composition to help further reduce oxidation and/or degradation of the HCA.

The antioxidant, when included, may be present at 0.001% to 3% by weight of the composition (e.g., 0.01% to 2%, 0.05% to 1%, or 0.1% to 0.5%). Some non-limiting examples of antioxidants that may be suitable for use herein are sodium sulfite, sodium bisulfite sodium metabisulfite, and butylated hydroxytoluene.

### Emulsifier

When the low-pH composition herein is in the form of an emulsion (e.g., oil-in-water emulsion), it may be desirable to include an emulsifier to stabilize the emulsion (i.e., prevent the emulsion from phase separating). The emulsifier may be present in the composition at 0.01% to 10% by weight of the composition (e.g., 0.05% to 5% or 0.1% to 2%). The emulsifiers may be nonionic, anionic or cationic. In some instances, the emulsifier may be a silicone emulsifier. Some non-limiting examples of emulsifiers that may be suitable for use herein are disclosed in U.S. Pat. Nos. 3,755,560; 4,421,769; and Mccutcheon's Detergents and Emulsifiers, North American Edition, pages 317-324 (1986).

Some other non-limiting examples of emulsifiers that may be suitable for use herein include ethers of polyglycols and of fatty alcohols, esters of polyglycols and of fatty acids, ethers of polyglycols and of fatty alcohols which are glycosylated, esters of polyglycols and of fatty acids which are glycosylated, ethers of C12-30 alcohols and of glycerol or of polyglycerol, esters of C12-30 fatty acids and of glycerol or of polyglycerol, ethers of oxyalkylene-modified C12-30 alcohols and of glycerol or polyglycerol, ethers of C1-230 fatty alcohols comprising and of sucrose or of glucose, esters of sucrose and of C1230 fatty acids, esters of pentaerythritol and of C12-30 fatty acids, esters of sorbitol and/or of sorbitan and of C12 30 fatty acids, ethers of sorbitol and/or of sorbitan and of alkoxylated sorbitan, ethers of polyglycols and of cholesterol, esters of C12-30 fatty acids and of alkoxylated ethers of sorbitol and/or sorbitan, and combinations thereof. A particularly useful class of emulsifiers is polyethylene glycol ethers of lauryl alcohol such as laureth-1 through laureth-50 (e.g., laureth-4). Still other examples of emulsifiers include ethers of glycerol, polyglycerol, sucrose, glucose, or sorbitol; esters of glycerol, polyglycerol, sucrose, glucose, or sorbitol; and mixtures thereof. Other particularly useful classes of emulsifiers are the alkyl esters of sorbitol and sorbitol anhydrides such as polysorbate 20, polysorbate 21, and polysorbate 40.

In some aspects, it may be desirable to include a linear or branched silicone emulsifier in the low-pH composition. Particularly useful silicone emulsifiers may include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin-Etsu. A particular suitable emulsifier for use herein is PEG-11 methyl ether dimethicone, which is available from Shin-Etsu as KF-6011. It was discovered that the PEG-11 methyl ether dimethicone emulsifier further reduced the tacky feel of the anionic polymer thickener, thereby improving the overall feel of the low-pH composition. The emulsifier may be present at an amount of 0.1% to 10% by weight of the composition (e.g., 1% to 5%, or 2% to 4%).

### Bakuchiol

Bakuchiol can be contained in the composition at a level of from about 0.01% to about 2%, preferably from about 0.1% to about 1%, in view of, for example, improving skin wrinkles, fine lines, skin texture, elasticity and firmness, and/or improving skin tone such as reducing dark spots and balancing uneven skin tone. It has been surprisingly found that, bakuchiol shows improved penetration to skin when bakuchiol is contained in the low pH formulations of the present disclosure, compared to neutral/alkaline pH formulations.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, antioxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition at low-pH, especially pH sensitive ingredients like niacinamide, salicylates and peptides. In some instances, it may be desirable to select skin care actives that function via different biological pathways so that the actives do not interfere with one another, which could reduce the efficacy of both agents. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The low-pH compositions herein are formulated for topical application to skin. The method of using the present low-pH composition involves identifying a target portion of skin on a person in need of treatment or where treatment is desired (e.g., portions of skin exhibiting uneven tone, sallowness, hyperpigmented spots, fine lines or wrinkles, skin stability) and applying an effective amount of the low-pH composition to the target portion of skin over the course of a treatment period. The effective amount of composition may vary based on the skin benefit desired by the user and/or the size of the treatment area. In some instances, the effective amount may range from 0.1 g to 5 g (e.g., 0.2 g to 4 g, 0.3 g to 2 g, or even 0.5 g to 1 g). The target portion of skin may be on a facial skin surface such as the forehead, perioral, chin, periorbital, nose, and/or cheek) or another part of the body (e.g., hands, arms, legs, back, chest). In some instances, a target portion of skin may be selected that does not currently exhibit signs of skin aging, such as hyperpigmented spots or uneven skin tone, but is an area of skin that commonly exhibits such features with age. In these instances, the low-pH composition may be used to help prevent the occurrence of such undesirable skin features.

The composition may be applied locally to the target portion of skin in need of treatment and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. When used according to the methods herein, the present compositions may improve the appearance and/or function of skin, for example, by improving skin texture. Improvements in skin texture can be provided, for example, by decreasing pore size, reducing skin roughness, reducing the presence and/or size of wrinkles, combinations of these and the like.

The treatment period is ideally of sufficient time for the low-pH composition to improve the appearance and/or function of the target portion of skin. The treatment period typically lasts for at least 1 week (e.g., about 2 weeks, 4 weeks, 8 weeks, or even 12 weeks). In some instances, the treatment period may extend over multiple months (i.e., 3-12 months). In some instances, the composition is applied most days of the week (e.g., at least 4, 5 or 6 days a week), at least once a day or even twice a day during a treatment period of at least 2 weeks, 4 weeks, 8 weeks, or 12 weeks.

The step of applying the composition herein may be accomplished by localized application. In reference to application of the composition, the terms “localized”, “local”, or “locally” mean that the composition is delivered to the targeted area (e.g., a psoriatic plaque) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Methods

### Color

This method can be used to determine the change in color of a product, material, or target portion of skin. A spectrophotometer (e.g., Spectrophotometer CM-3600A, Konica Minolta, Japan, or equivalent) is used to measure spectrometric data for CIE (International Commission on Illumination) specified illuminating and viewing conditions, and compute the associated tristimulus values, XYZ, based on CIE observer and a CIE illuminant standards. The ASTM standard for obtaining spectrometric data for Object-Color Evaluation is ASTM E1164-12 (2017) el with the values and procedures for computing CIE tristimulus values from spectrometric data outlined in ASTM E308. The CIELAB color scale, also referred to as L*a*b*, are calculated from the tristimulus values as defined in ASTM E308. The L* denotes the sample's perceptual lightness. Whereas a* and b* relate to the unique colors of human vision, with a* being positive in the red direction and negative in the green direction, and b* positive in the yellow direction and negative in the blue direction.

The spectrophotometer is operated with a 2° observer and D65 illuminant to measure 1931 CIE defined tristimulus XYZ values with and associated CIELAB colors. Portion samples are evaluated using a 10 mm path length in a plastic cell (e.g., CM-A131, Konica Minolta, Japan or equivalent), reflectance measurement, a 25.4 mm aperture opening at the specimen surface, and specular component excluded conditions with a standard white background. Acceptable white backgrounds include the white portion of an opacity card or equivalent (e.g., opacity card Form 2A, Leneta Company, Inc, Mahwah, NJ). The instrument is calibrated with a zero standard, utilizing a zero-calibration box, or black trap, that is free from dust and debris (e.g., Konica Minolta zero calibration box CM-A155 or equivalent). The CIELAB values are reported by the associated instrument software (e.g., SpectroMagic NX) as defined in ASTM E308. For changes in product yellowness, the CIELAB color scale is utilized focused on the change in the b* value. The results are reported as a Δb*, where a positive Δb* indicates increased yellowness.

### Rheology

This method provides a way to measure the dynamic viscosity of a composition or material using a BROOKFIELD brand viscometer (e.g., model DV2T or equivalent) and a suitable spindle (e.g., RV4 or equivalent) according to the manufacturer's instructions. It is to be appreciated that the skilled artisan will be able to select the appropriate spindle in accordance with the manufacturer's recommendation. After calibrating the viscometer, the spindle is immersed into a sufficient quantity of test sample (e.g., enough to immerse the spindle up to the immersion mark on the spindle shaft). Set the spindle rotation speed to 5 rpm, and then start the viscometer. Allow time for the indicated viscosity reading to stabilize (approximately 10-30 seconds). After the reading stabilizes, take 5 readings at 10 second intervals. Calculate the viscosity as the average of the 5 readings.

### Examples

The compositions in below Table 1 and 2 can be prepared using conventional methods of making skin care compositions. Such methods typically involve mixing of the ingredients in one or more steps to a relatively uniform state, with or without heating, cooling, application of vacuum, and the like. All exemplified amounts exclude minor materials such as diluents, preservatives, color solutions, feel modifying powders and elastomers, etc., that may be present in a commercial product unless otherwise specified.

When HCA is included, HCA may be added as a solid form and solubilized in-situ, dissolved as a premix, or supplied as a pre-dispersed raw material. Such premix may be a premix of HCA, polar emollient, and co-solvent for HCA. All other materials are listed ‘as is’ from the suppliers and not broken down into the individual constituents.

TABLE 1
Formulations

| | CEx. A | Ex. 1 | CEx. B |
|---|---|---|---|
| L Lactic Acid | 1.62% | 0.495% | 0.000% |
| Sodium Lactate | 1.17% | 0.000% | 0.000% |
| Glucono-deltalactone | 0.000% | 1.990% | 3.100% |
| Sodium Gluconate | 0.000% | 1.800% | 1.800% |
| Water Purified | q.s. | q.s. | q.s. |
| | to 100% | to 100% | to 100% |
| Glycerin | 1.900% | 1.900% | 1.900% |
| Dimethicone 5 cst *1 | 1.900% | 1.900% | 1.900% |
| Niacinamide | 2.000% | 2.000% | 2.000% |
| Polyacrylate | 1.100% | 1.100% | 1.100% |
| Crosspolymer-6 *2 | | | |
| Panthenol (D-Panthenol | 0.500% | 0.500% | 0.500% |
| Disodium EDTA | 0.100% | 0.100% | 0.100% |
| PEG-11 Methylether | 10.100% | 0.100% | 0.100% |
| Dimethicone *3 | | | |
| Trehalose | 1.500% | 1.500% | 1.500% |
| Sodium Benzoate | 0.050% | 0.050% | 0.050% |
| Pentylene Glycol | 1.900% | 1.900% | 1.900% |
| Lipobrite HCA-4 | 2.000% | 2.000% | 2.000% |
| (pCoumaric acid) *4 | | | |
| Isopropyl Lauroyl | 1.900% | 1.900% | 1.900% |
| Sarcosinate *5 | | | |
| Sodium Sulfite | 0.050% | 0.050% | 0.050% |
| Ethyl Ascorbic Acid | 0.110% | 0.110% | 0.110% |
| Perfume | 0.100% | 0.100% | 0.100% |
| Nymphaea alba | 0.400% | 0.400% | 0.400% |
| Flower Extract | | | |
| Sorbitan Stearate | 0.300% | 0.300% | 0.300% |
| PROPYLENE GLYCOL | 1.900% | 1.900% | 1.900% |
| Dipropylene Glycol | 1.900% | 1.900% | 1.900% |
| Lauroyl Lysine | 2.000% | 2.000% | 2.000% |
| Boron Nitride | 0.800% | 0.800% | 0.800% |
| Mica and Titanium Dioxide | 0.600% | 0.600% | 0.600% |
| Synthetic Mica and TiO2 | 0.400% | 0.400% | 0.400% |
| TOTALS: | 100.00% | 100.00% | 100.00% |
| pH | 3.8 | 3.8 | 3.8 |

*1 DC-1503 from Dow Corning
*2 SEPIMAX ZEN available from Seppic
*3 KF-6011 available from Shin-Etsu
*4 Lipobrite ®: from Vantage, a pre-dispersed 15% solution of 4-HCA in PEG-4
*5 Eldew SL-205 from Ajinomoto OmniChem

Ex. 1 is an example of the present discosure. CEx. A (using a buffering system comprising lactic acid, but without PHA and its buffering salt) and CEx.B (using a buffering system comprising PHA and its buffering salt, but without lactic acid) are comparative examples.

The composition of the present disclosure provides reduced skin irritation and/or improved skin barrier function (such as transepidermal water loss (TEWL)), while providing skin care efficacy/improving skin condition and/or stability of the composition. Some benefits of such compositions can be also understood by the following data.

### TEWL Test Method and Result

34 Chinese Female test subjects aged 18-45 were selected for testing a 2-week treatment study for trans-epidermal water loss (TEWL) on the dorsal forearm. The study included 2-week test period with 3 product treatment locations randomized with a maximum of 2 per arm (3×4 cm2 per site, 1 or 2 from left dorsal forearm and 1 or 2 from right dorsal forearm) were assigned on each panelist. The panelists were instructed to apply Approximately 2 ul/cm2 of each product to designed site twice per day (morning/evening) during the test period. 3 replicates of TEWL measurement per site were taken with VapoMeter (Delfin Technologies) at each time points (baseline, 1-week, 2-week). The change in TEWL was measured at each timepoint with final results for the 2-week TEWL change from baseline.

2 Week TEWL Results are shown in below table.

| | 2 WK Delta TEWL | |
|---|---|---|
| | (Change from | Statistical |
| Formulations | Baseline) | Grouping |
| CEx. A | 0.44 | a |
| Ex. 1 | −0.32 | b |
| CEx. B | −0.58 | b |

Statistical grouping means that, for the test group a is significantly different than test group b (pair-wise t-test comparisons with significance of 0.1). This means that Ex. 1 and CEx. B have significantly better TEWL results compared to CEx. A, thus, Ex. 1 and CEx. B have significantly better skin barrier function compared to CEx. A.

### Photostability Test Method and Result

The formulations of Ex. 1, CEx. A, and CEx. B were exposed to a minimum of 6,800,000 lux accumulated of visible light with a controlled photo-stability light chamber (such as the Aralab Fitoclima 600 PLH-R, or similar equipment). The units of illuminance in lux, is defined as 1 lux is equivalent to 1 lumen per square meter. Accumulated lux, also sometimes called lux hours, are the calculated as the lux per hour multiplied by the exposure time of the photo-stability test. The Aralab, Fitoclima 600 PLH-R is equipped with 8 fluorescent HF ‘cool white’ lamps. Product samples were tested in glass containers approximately 7.5 centimeters from the fluorescent lamps. The photo-stability test was conducted at an accelerated condition of 26K lux/hr for approximately 11 days with no UV light exposure at 25° C. The color was measured at the start of test and after photo-exposure per the ‘color’ method described previously. The change in yellowness, delta b*, is reported for the stability test for each formula tested.

Photo stability result is shown in below table.

| Formulations | Color (Delta B) db |
|---|---|
| CEx. A | 2.69 |
| Ex. 1 | 0.1 |
| CEx. B | 0.22 |

Ex. 1 have improved photostability, compared to CEx. A and CEx. B.

TABLE 2
Formulations

| Component | A | B | C | D | E | F | G |
|---|---|---|---|---|---|---|---|
| Water | qs to 100% | | | | | | |
| Glycerin | 7.00 | 3.00 | 3.00 | 3.00 | 3.00 | 3.00 | 3.00 |
| Dimethicone 5 cSt | — | — | 2.00 | 2.00 | 2.00 | 2.00 | 2.00 |
| Niacinamide | 2.00 | 2.00 | 2.00 | 2.00 | 5.00 | 5.00 | 5.00 |
| Gluconic Acid | 1.00 | 2.00 | — | — | 2.30 | — | — |
| Sodium Gluconate | 1.00 | 2.5 | 1.0 | 1.0 | 3.00 | 2.00 | 2.00 |
| Lactobionic Acid | — | 1 | 1.8 | — | — | — | 1.50 |
| Maltobionic Acid | — | — | — | 1.8 | — | 1.50 | — |
| Lactic acid | 0.30 | 1.0 | 0.5 | 0.5 | 0.50 | 0.5 | 0.50 |
| Sodium lactate | 0.30 | — | — | — | — | — | — |
| Polyacrylate crosspolymer-6 ¹ | — | — | 1.25 | 1.25 | 1.25 | 1.25 | 1.25 |
| Sodium polyacryloyldimethyl taurate ² | 1.25 | 1.25 | — | — | — | — | — |
| D-Panthenol | 0.50 | 0.50 | 0.50 | 0.50 | 0.50 | 0.50 | 0.50 |
| Disodium EDTA | 0.10 | 0.10 | 0.10 | 0.10 | 0.10 | 0.10 | 0.10 |
| PEG-11 methyl ether dimethicone ³ | — | — | 0.10 | 0.10 | 0.10 | 0.10 | 0.10 |
| Trehalose | — | — | 0.50 | 0.50 | 0.50 | 0.50 | 0.50 |
| Sodium benzoate | 0.05 | 0.05 | 0.05 | 0.05 | 0.05 | 0.05 | 0.05 |
| Sodium sulfite | — | 0.05 | 0.05 | 0.05 | 0.05 | 0.05 | 0.05 |
| 4-HCA ⁴ | 0.0001 | 1.00 | 0.50 | 0.25 | 0.10 | 1.00 | 0.30 |
| PEG-4 | 0.00057 | 5.67 | 2.83 | 1.42 | 0.57 | 5.67 | 1.7 |
| Isopropyl lauroyl sarcosinate ⁵ | 0.10 | 5.00 | 4.00 | 3.00 | 1.00 | 5.00 | 1.00 |
| Pentylene glycol | 0.10 | 3.00 | 3.00 | 3.00 | 3.00 | 3.00 | 3.00 |
| Propylene glycol | — | 5.00 | — | — | — | 5.00 | 5.00 |
| Dipropylene glycol | — | 4.00 | — | — | — | 4.00 | 4.00 |
| Ethoxydiglycol | — | 2.60 | — | — | — | 2.60 | — |
| Isohexadecane | 3.00 | 3.00 | — | — | — | — | — |
| Isopropyl isostearate | 1.50 | 1.50 | — | — | — | — | — |
| Cetearyl glucoside and cetearyl | 0.50 | 0.50 | — | — | — | — | — |
| Behenyl Alcohol | 0.70 | 0.70 | — | — | — | — | — |
| Stearyl Alcohol | 1.20 | 1.20 | — | — | — | — | — |
| Cetyl Alcohol | 0.90 | 0.90 | — | — | — | — | — |
| PEG-100 Stearate | 0.10 | 0.10 | — | — | — | — | — |
| Stearic Acid | 0.10 | 0.10 | — | — | — | — | — |
| Dimethicone and dimethiconol ⁶ | 1.00 | 1.00 | — | — | — | — | — |
| Palmitoyl pentapeptide-4 ⁷ | 0.50 | 0.50 | — | — | — | — | — |
| Bakuchiol | 0.1 | 1.0 | — | — | — | — | — |
| NaOH | * pH adjustment as necessary | | | | | | |
| HCl | * pH adjustment as necessary | | | | | | |
| pH | 4.0 | 4.0 | 4.0 | 4.0 | 3.0 | 3.5 | 3.8 |
| Ratio** (PHA + PHA salt):(lactic | 2:0.3 | 5.5:1 | 2.8:0.5 | 2.8:0.5 | 5.3:0.5 | 3.5:0.5 | 3.5:0.5 |
| acid) | | | | | | | |
| Ratio** (PHA):(lactic acid) | 1:0.3 | 3:1 | 1.8:0.5 | 1.8:0.5 | 2.3:0.5 | 1.5:0.5 | 1.5:0.5 |
| Ratio** (PHA salt):(lactic acid) | 1:0.3 | 2.5:1 | 1:0.5 | 1:0.5 | 3:0.5 | 2:0.5 | 2:0.5 |

**Ratio calculated based on material added ‘as-is’ to composition
¹ SEPIMAX ZEN available from Seppic
² ARISTOFLEX SILK available from Clariant
³ KF-6011 available from Shin-Etsu
⁴ LIPOBRITE from Vantage
⁵ Eldew SL-205 from Ajinomoto OmniChem
⁶ DC-1503 from Dow Corning
⁷ PROMATRIXYL from Croda

Compositions A-G in the above Table 2 are also examples of the present disclosure. The composition of the present disclosure provides reduced skin irritation and/or improved skin barrier function (such as transepidermal water loss (TEWL)), while providing skin care efficacy/improving skin condition and/or stability of the composition.

### Example Combinations

1. A low-pH, aqueous skin care composition, comprising: (a) Lactic acid; (b) A polyhydroxy acid (PHA); (c) A buffering salt of the polyhydroxy acid (PHA); and (d) Water, wherein the pH of the composition is from about 2.5 to 4.5, wherein the composition meets at least one of the following conditions (i)-(iii):
(i) wherein the weight ratio between “the sum of (b) PHA and (c) PHA buffering salt” and (a) lactic acid is from about 14:1 to about 1:1, preferably from about 12:1 to about 2:1, more preferably from about 10:1 to about 5:1;
(ii) wherein the weight ratio between (b) PHA and (a) Lactic acid is from about 10:1 to about 2:1, preferably from about 7:1 to about 2:1, more preferably from about 5:1 to about 3:1;
(iii) wherein the weight ratio between (c) PHA buffering salt and (a) Lactic acid is from about 10:1 to about 2:1, preferably from about 7:1 to about 2:1, more preferably from about 5:1 to about 2.5:1.
2. The composition of the preceding feature, wherein (b) the polyhydroxy acid (PHA) is selected from the group consisting of Gluconic acid, Glucono-deltalactone (GDL), Lactobionic acid, Maltobionic acid, and mixtures thereof.
3. The composition of any of the preceding features, wherein (b) the polyhydroxy acid (PHA) is Gluconic acid, Glucono-deltalactone (GDL) and mixtures thereof, preferably Glucono-deltalactone (GDL).
4. The composition of any of the preceding features, wherein (c) the PHA buffering salt is Sodium Gluconate.
5. The composition of any of the preceding features, further comprising from about 0.1% to about 10% of a hydroxycinnamic acid (HCA), preferably wherein the HCA is p-coumaric acid.
6. The composition of any of the preceding features, further comprising from about 0.1% to about 10% of a polar emollient, with the weight ratio between HCA and the polar emollient is from about 1:1 to about 1:10.
7. The composition of any of the preceding features, further comprising from about 1.5% to less than 20% of a co-solvent for HCA, with a Hansen solubility parameter distance of less than 15 from hydroxycinnamic acid (HCA).
8. The composition of any of the preceding features, wherein the co-solvent is a glycol selected from the group consisting of propylene glycol, dipropylene glycol, butylene glycol, pentylene glycol, hexylene glycol, ethoxydiglycol, C2-C6 polyethene glycols, and combinations thereof.
9. The composition of any of the preceding features, wherein the composition further comprises a mica.
10. The composition of any of the preceding features, further comprising from about 0.1% to about 10% of a vitamin B₃ compound.
11. The composition of any of the preceding features, further comprising from about 0.01% to about 1% of a bakuchiol.
12. A method of improving penetration of bakuchiol, by using the composition of the preceding feature 11.
13. A method of cosmetically treating skin, comprising:
identifying a target portion of skin where treatment is desired; and
applying an effective amount of the composition of any of the preceding features to the target portion of skin over the course of a treatment period.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A low-pH, aqueous skin care composition, comprising:
(a) lactic acid;
(b) a polyhydroxy acid (PHA);
(c) a buffering salt of the polyhydroxy acid (PHA); and
(d) water;
wherein the pH of the composition is from about 2.5 to about 4.5;
wherein the composition meets at least one of the following conditions (i)-(iii):
(i) wherein the weight ratio between “the sum of (b) PHA and (c) PHA buffering salt” and (a) lactic acid is from about 14:1 to about 1:1;
(ii) wherein the weight ratio between (b) PHA and (a) Lactic acid is from about 10:1 to about 2:1;
(iii) wherein the weight ratio between (c) PHA buffering salt and (a) Lactic acid is from about 10:1 to about 2:1.

2. The composition of claim 1, wherein the weight ratio between “the sum of (b) PHA and (c) PHA buffering salt” and (a) lactic acid is from about 12:1 to about 2:1.

3. The composition of claim 2, wherein the weight ratio between “the sum of (b) PHA and (c) PHA buffering salt” and (a) lactic acid is from about 10:1 to about 5:1.

4. The composition of claim 1, wherein the weight ratio between (b) PHA and (a) lactic acid is from about 7:1 to about 2:1.

5. The composition of claim 4, wherein the weight ratio between (b) PHA and (a) lactic acid is from about 5:1 to about 3:1.

6. The composition of claim 1, wherein the weight ratio between (c) PHA buffering salt and (a) lactic acid is from about 7:1 to about 2:1.

7. The composition of claim 6, wherein the weight ratio between (c) PHA buffering salt and (a) lactic acid is from about 5:1 to about 2.5:1.

8. The composition of claim 1, wherein (b) the polyhydroxy acid (PHA) is chosen from gluconic acid, glucono-deltalactone (GDL), lactobionic acid, maltobionic acid, or mixtures thereof.

9. The composition of claim 8, wherein (b) the polyhydroxy acid (PHA) is gluconic acid and/or glucono-deltalactone (GDL).

10. The composition of claim 9, wherein (b) the polyhydroxy acid (PHA) is glucono-deltalactone (GDL).

11. The composition of claim 1, wherein (c) the PHA buffering salt is sodium gluconate.

12. The composition of claim 1, further comprising from about 0.1% to about 10% of a hydroxycinnamic acid (HCA).

13. The composition of claim 12, wherein the HCA is p-coumaric acid.

14. The composition of claim 12, further comprising from about 0.1% to about 10% of a polar emollient, with the weight ratio between HCA and the polar emollient is from about 1:1 to about 1:10.

15. The composition of claim 12, further comprising from about 1.5% to less than 20% of a co-solvent for HCA, with a Hansen solubility parameter distance of less than 15 from hydroxycinnamic acid (HCA).

16. The composition of claim 15, wherein the co-solvent is a glycol chosen from propylene glycol, dipropylene glycol, butylene glycol, pentylene glycol, hexylene glycol, ethoxydiglycol, C2-C6 polyethene glycols, or mixtures thereof.

17. The composition of claim 1, wherein the composition further comprises a mica.

18. The composition of claim 1, further comprising from about 0.1% to about 10% of a vitamin B₃ compound.

19. The composition of claim 1, further comprising from about 0.01% to about 1% of a bakuchiol.

20. A method of improving penetration of bakuchiol, by using the composition of claim 19.

21. A method of cosmetically treating skin, comprising:
identifying a target portion of skin where treatment is desired; and
applying an effective amount of the composition of claim 1 to the target portion of skin over the course of a treatment period.
